(19) United States
(12) Patent Application Publication
(10) Pub. No.: US 2011/0066027 A1
(43) Pub. Date: Mar. 17, 2011

(54) Combined Imaging System with a Magnetic Resonance System and a UWB Radar

(76) Inventor: Wilfried Schnell, Forchheim (DE)

(21) Appl. No.: 12/879,061

(22) Filed: Sep. 10, 2010

(30) Foreign Application Priority Data
Sep. 11, 2009 (DE) 10 2009 041 261.1

(51) Int. Cl. A61B 5/055

(52) U.S. Cl. 600/411

(57) ABSTRACT

During operation of a combined imaging system with a magnetic resonance system and a UWB radar, a wireless signal transmission in conjunction with a patient monitoring system is to be enabled in a simpler and more cost-effective manner. To this end, the use of already existing components of the UWB radar is proposed for the wireless signal transmission. This makes it possible to dispense with an additional transmitting and/or receiving antenna for the wireless signal transmission.

### CROSS REFERENCE TO RELATED APPLICATIONS

This application claims priority of German application No. 10 2009 041 261.1 filed Sep. 11, 2009, which is incorporated by reference herein in its entirety.

### FIELD OF THE INVENTION

The invention relates to a combined imaging system, comprising a magnetic resonance system and a UWB radar, to produce images of an examination area of an examination object which is located in a recording room of the imaging system. In addition, the invention relates to a process for operating such a combined imaging system.

### BACKGROUND OF THE INVENTION

Magnetic resonance tomography, also referred to as nuclear spin tomography, is a technique now in widespread use for acquiring images from inside the body of a living examination object. In order to produce an image with this technique, i.e. a magnetic resonance recording of an examination object, the body or the part of the patient's body being examined must be exposed initially to a basic magnetic field which is as homogeneous as possible (mostly referred to as the B₀ field), which is produced by a basic field magnet of the magnetic resonance system. During the recording of the magnetic resonance images, rapidly switched gradient fields for location coding, which are produced by gradient coils, are superimposed on this basic magnetic field. In addition, HF signals, for example a high-frequency pulse or a high-frequency pulse sequence, of a defined field strength are radiated by a high-frequency antenna into the examination volume in which the examination object is located. By means of this HF field (mostly referred to as the B₁ field) the nuclear spins of the atoms in the examination object are excited such that they are deflected from their balanced position running parallel to the basic magnetic field and rotate around the direction of the basic magnetic field. The magnetic resonance signals caused by this are captured by high-frequency receiving antennas. The receiving antennas can either be the same antennas used for emitting the high-frequency pulses or separate receiving antennas. The magnetic resonance images of the examination object are then produced on the basis of the magnetic resonance signals received. Each pixel in the magnetic resonance image is assigned to a small body volume, a “voxel”, and each brightness or intensity value of the pixels is connected with the signal amplitude of the magnetic resonance signal received from this voxel.

A magnetic resonance system, which is part of a combined imaging system in accordance with the invention, is e.g. known from patent application DE 10 2007 057 495 A1.

Furthermore, a medical imaging technique designated as “UWB radar” (ultra-wideband radar) is known, with which likewise images of a living examination object can be produced. In this method the examination object is irradiated with wideband electromagnetic pulses of low power which penetrate into the examination object and are partially reflected on the boundary layers of tissue types having different dielectric properties. The UWB signal is generated by a UWB signal generator and radiated into the examination room by an antenna. A receiving antenna in connection with a receiver device then receives a UWB echo signal from different depths of the examination object. Through a shift of the boundary layers between the tissue types caused by respiration and heartbeat it is possible to present these biological events.

From publication U.S. Pat. No. 5,668,555 a UWB radar is known for examining an examination object, by which a sequence of pulses is transmitted to the examination object and the UWB echo signals then reflected by the examination object are received and analyzed. Also on the basis of this measurement principle it is possible to produce image data of the examination object.

A combination of the two mentioned methods to form a combined imaging system is known from publication F. Thiel, M. A. Hein, J. Sachs, U. Schwarz, F. Seifert: “Physiological signatures monitored by ultra-wideband-radar validated by magnetic resonance imaging”, Proc IEEE ICUWB 2008, vol. 1, pages 105-108.

Through the combination of the two imaging techniques it becomes possible to conduct examinations on living examination objects in real time and, for example, to present the heartbeat or respiration.

From telecommunications engineering and the term “UWB communication” (“ultra wideband communication”) close-range radio communication by transmission of short pulses is known (“pulse radio”). To transmit the signal, an extremely large frequency range is used with a bandwidth of at least 500 MHz or at least 20% of the arithmetical mean value of the lower and upper threshold frequency of the frequency band used.

From patent document EP 1 719 256 B1 a communication system is known in which wireless signal transmission is based on ultra-wideband (UWB) pulse technology.

In a magnetic resonance examination it is frequently necessary to capture other signals or data from the recording room in addition to the magnetic resonance signals or to transfer data or signals to the recording room. This makes it possible for example to monitor the patient (pulse, blood pressure, etc.) or to communicate with the patient during an MR examination. For signal transmission it is possible to use MR compatible lines (e.g. optical waveguides etc.). Their use, however, makes it difficult to prepare and therefore delays the examination.

To avoid cable connections, wireless transmission techniques are also used, such as for example are known from entertainment electronics. For use in connection with a magnetic resonance system, however, the known transmission techniques have to be adapted. Altogether the provision of an MR compatible transmission system increases the total costs of a magnetic resonance system equipped with such.

From publication US 2005/0107681 A1 a patient monitoring system is known in which body signals of a patient are captured during a magnetic resonance examination and transmitted wirelessly to a receiver device of the patient monitoring system located outside the recording room of the magnetic resonance system.

A disadvantage of patient monitoring during a magnetic resonance recording is the high technical effort which has to be made so that the patient monitoring system does not negatively influence the magnetic resonance recording and itself is not adversely affected by the magnetic resonance system.

### SUMMARY OF THE INVENTION

The object of the present invention is to simplify in a combined imaging system wireless signal transmission between a unit located in the recording room of the imaging system and an external unit.

This object is achieved by a combined imaging system and by a process for operating a combined imaging system with the features in accordance with the claims.

The combined imaging system comprises a magnetic resonance system and a UWB radar, to produce images of an examination area of an examination object which is located in a recording room of the imaging system, whereby the magnetic resonance system comprises a high-frequency device for transmitting high-frequency signals into the examination area and for receiving magnetic resonance signals then emitted from the examination area, whereby the UWB radar comprises a UWB transmitter device for transmitting UWB signals into the examination area and a UWB receiver device for receiving UWB echo signals then reflected from the examination area, and whereby imaging means for producing image data of the examination area based on the magnetic resonance signals and the UWB echo signals are present.

In accordance with the invention at least one unit is located in the recording room of the combined imaging system, for example a sensor which records physical conditions (pulse rate, blood pressure, oxygen saturation, etc.) of the patient being examined. In addition, the unit can facilitate communication of the patient with the examination personnel. For this purpose, the unit is configured e.g. as a simple nurse call device or as a more convenient headset. Usually there are several such units in the recording room. The invention facilitates in a simple way wireless signal transmission from and/or to such a unit. At least components of the UWB radar, for example an antenna, are advantageously used, both for imaging by means of the UWB radar as well as for signal transmission from/to the unit. This enables the additional costs for wireless signal transmission in connection with the imaging system to be reduced. In the ideal case a separate wireless signal transmission system can largely be dispensed with, as the UWB radar takes over this function. No further antenna other than the antenna in any case required for the UWB radar needs to be installed on the imaging system for wireless signal transmission to/from the unit. This reduces the total cost of the imaging system.

### BRIEF DESCRIPTION OF THE DRAWINGS

The invention is explained below with reference to an exemplary embodiment, whereby:

FIG. 1 is a very simplified schematic representation of a combined imaging system with a magnetic resonance system as well as a UWB radar, and

FIG. 2 is a schematic representation of a headset.

### DETAILED DESCRIPTION OF THE INVENTION

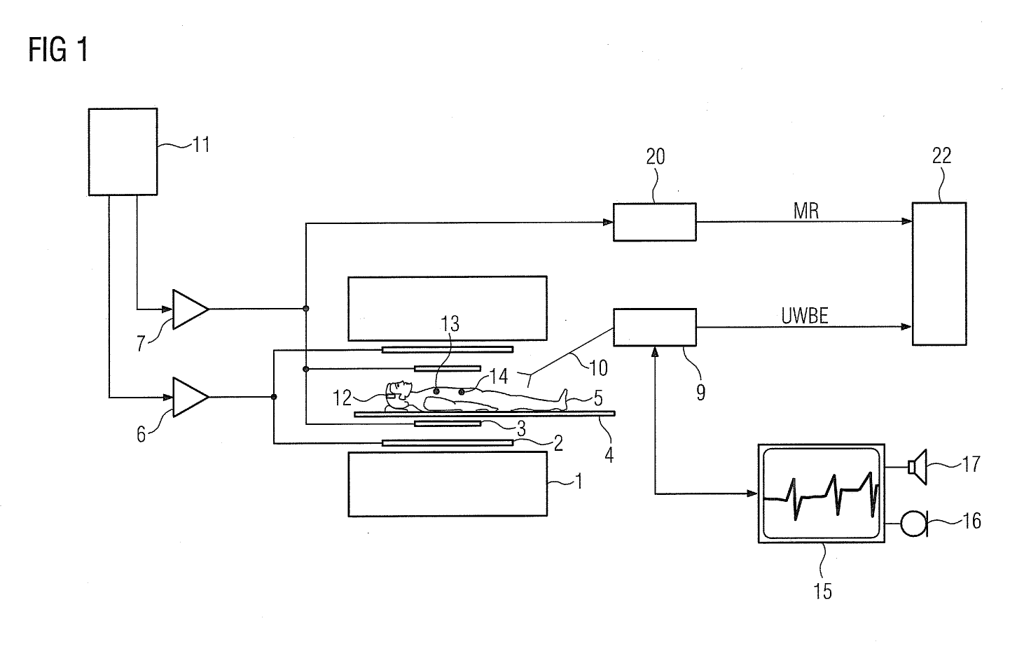

FIG. 1 shows in a very simplified schematic representation the main components of a combined imaging system which comprises a magnetic resonance system and a UWB radar 9. The magnetic resonance system exhibits an essentially cylindrically hollow base unit 1, in whose cavity a patient recording room (a gantry) is located. A patient positioning device 4, on which a patient 5 to be examined lies, can be moved into the gantry. To examine the patient 5 with the magnetic resonance system, an electrical gradient pulse signal G is generated and amplified by means of a signal generator 11 and a gradient pulse amplifier 6 and sent to the gradient coils 2. These then emit rapidly switched gradient fields. Electrical high-frequency pulse signals RF are also generated and amplified by the signal generator 11 and a high-frequency pulse amplifier 7 and sent to the high-frequency pulse antennas. These then radiate high-frequency fields. As a result of this excitation the patient 5 emits magnetic resonance signals which are picked up by the high-frequency pulse antennas 3 and sent to an MR receiver unit 20. The latter converts the received signals into an electrical MR signal.

In addition, the combined imaging system comprises a UWB radar 9 with a transmitting and receiving antenna 10. In a UWB transmitter device (not shown) the UWB radar 9 generates a UWB signal and transmits this into the examination room via the transmitting and receiving antenna 10. Likewise via the transmitting and receiving antenna 10, in a UWB receiver device (not shown) of the UWB radar 9 a resulting UWB echo signal from the examination room is captured and after further processing in the UWR radar 9 an electrical UWB echo signal UWBE is generated. In the exemplary embodiment the UWB radar 9 comprises only an antenna 10, which is part both of the transmitter as well as of the receiver device. Several antennas can, however, be present, in particular a transmitting and a receiving antenna.

The UWBE and MR signals are then sent for further processing and in particular for image generation to a signal processing unit 22, which calculates image data from the signals.

In addition, the combined imaging system includes a patient monitoring system. This comprises a headset worn on the head of the patient 5, by which the patient 5 can talk to a person outside the recording room. The system also includes sensors 13 and 14, with which the pulse rate and the oxygen saturation of the blood can be measured. Signal transmission to and from the units 12, 13 and 14 takes place wirelessly. For this purpose the headset 12 is fitted with a transmitter and receiver device and the sensors 13 and 14 with transmitter devices. Ultra-wideband (UWB) technology is used for signal transmission in accordance with the invention. Components of the UWB radar are advantageously used for the requisite transmitter and receiver unit outside the recording room. In particular, wireless signal transmission with the units 12-14 also takes place via the transmitting and receiving antenna 10 of the UWB radar. The antenna 10 is, for example, integrated in the base unit 1 of the magnetic resonance system. A further antenna for wireless signal transmission with the headset 12 or the sensors 13 and 14 is not required.

In addition to units 12-14 the patient monitoring system also comprises the user interface 15, in which signal processing takes place and the data captured by sensors 13 and 14 can be visualized. The user interface 15 comprises a microphone 16 and a loudspeaker 17 for communication with the patient 5 in connection with the headset 12.

Several transmission channels of UWB communication can be realized by corresponding modulation of the UWB signals. Pulse position or pulse duration modulation mainly come into consideration for this. Other processes are also known, however, for example coding through the polarity of the pulses, their amplitude or the use of orthogonal pulses. In particular by modulation or coding it is possible to differentiate pulses for image generation or for communication.

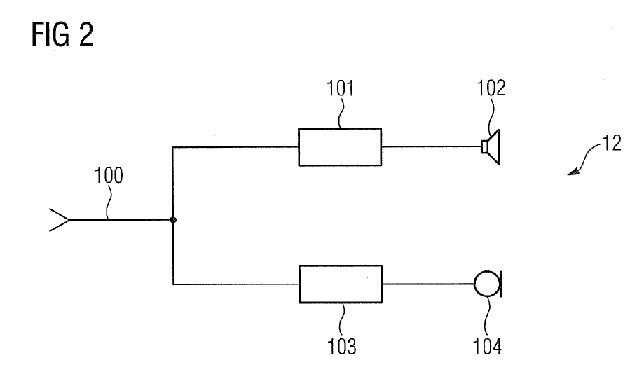

FIG. 2 shows in a very simplified schematic representation the headset 12 equipped for wireless signal transmission by means of UWB technology. For capturing an acoustic input signal this comprises a microphone 104 and for emitting an acoustic output signal a loudspeaker 102. For wireless signal transmission between the headset 12 and the user interface 15 a microphone signal emanating from the acoustic input signal is modulated in a modulator 103 and emitted via the antenna 100. The emitted signal thus reaches the antenna 10 of the UWB radar 9 wirelessly and is demodulated by the UWB radar 9 and sent to the user interface 15, where after signal processing it is emitted as an acoustic signal by the loudspeaker 17.

The signal transmission from the user interface 15 to the headset 12 takes place the other way round. Firstly an acoustic signal is picked up by the microphone 16 and after signal processing is sent to the UWB radar 9. After modulation the signal is emitted via the antenna 10 and picked up by the antenna 100 of the headset 12. Then the signal is demodulated by the demodulator 101, after which it is sent to a loudspeaker 102 for generation of an acoustic output signal.

The double function of components of the UWB radar 9 for image processing and for wireless signal transmission between various modules reduces the additional expense for wireless signal transmission in the combined imaging system to a minimum.

Advantageously the energy needed for operation of the headset 12, in particular for modulation and demodulation, is taken from an energy harvesting system. This preferably obtains its energy from the UWB transmission field.

1.-7. (canceled)

8. A combined imaging system for generating image data of an examination area of a patient located in a recording room of the combined imaging system, comprising:
a magnetic resonance system comprising a high-frequency device for transmitting a high-frequency signal into the examination area and for receiving a magnetic resonance signal emitted from the examination area;
a UWB radar comprising a UWB transmitter device for transmitting a UWB signal into the examination area and a UWB receiver device for receiving a UWB echo signal reflected from the examination area;
a signal processing unit for generating the image data of the examination area based on the magnetic resonance signal and the UWB echo signal; and
a unit located in the recording room for generating a signal being wirelessly transmitted between the UWB radar and the unit.

9. The combined imaging system as claimed in claim 8, wherein the unit is a headset worn on head of the patient.

10. The combined imaging system as claimed in claim 8, wherein the unit is a sensor arranged on body of the patient.

11. The combined imaging system as claimed in claim 10, wherein the sensor is a part of a patient monitoring system.

12. The combined imaging system as claimed in claim 11, wherein the sensor monitors a pulse rate of the patient.

13. The combined imaging system as claimed in claim 11, wherein the sensor monitors blood oxygen saturation of the patient.

14. The combined imaging system as claimed in claim 8, wherein the unit is an emergency call device.

15. The combined imaging system as claimed in claim 8, wherein the UWB transmitter device and the UWB receiver device is integrated into a transmitting and receiving antenna of the UWB radar.

16. The combined imaging system as claimed in claim 15, wherein the signal generated by the unit is wirelessly transmitted between the transmitting and receiving antenna of the UWB radar and the unit.

17. A process for operating a combined imaging system comprising a magnetic resonance system and a UWB radar for generating image data of an examination area of a patient located in a recording room of the combined imaging system, comprising:
transmitting a high-frequency signal into the examination area by the magnetic resonance system;
receiving a magnetic resonance signal emitted from the examination area by the magnetic resonance system;
transmitting a UWB signal into the examination area by the UWB radar;
receiving a UWB echo signal reflected from the examination area by the UWB radar;
generating the image data of the examination area based on the magnetic resonance signal and the UWB echo signal by a signal processing unit; and
wirelessly transmitting a signal generated by a unit located in the recording room between the UWB radar and the unit.

18. The process as claimed in claim 17, wherein the unit is a headset worn on head of the patient.

19. The process as claimed in claim 17, wherein the unit is a sensor arranged on body of the patient.

20. The process as claimed in claim 19, wherein the sensor is a part of a patient monitoring system.

21. The process as claimed in claim 20, wherein the sensor monitors a pulse rate of the patient.

22. The process as claimed in claim 20, wherein the sensor monitors blood oxygen saturation of the patient.

23. The process as claimed in claim 17, wherein the unit is an emergency call device.
